(19) United States
(12) Patent Application Publication
(10) Pub. No.: US 2022/0071891 A1
(43) Pub. Date: Mar. 10, 2022

(54) Extract of Chlamydomonas Acidophila, Method for Preparing Same and Cosmetic Compositions and Dermatological Compositions Comprising Same

(71) Applicant: Laboratoires Expanscience, Paris La Defense Cedex (FR)

(72) Inventors: Sophie LECLERE-BIENFAIT, Dreux (FR); Stephanie BREDIF, Chaudon (FR)

(73) Assignee: Laboratoires Expanscience, Paris La Defense Cedex (FR)

(21) Appl. No.: 17/416,868

(22) PCT Filed: Dec. 30, 2019
(86) PCT No.: PCT/EP2019/087171

§ 371 Date: Nov. 19, 2021

(30) Foreign Application Priority Data
Dec. 28, 2018 (FR) 1874321

(51) Int. Cl. A61K 8/9722; A61Q 19/00

(52) U.S. Cl. CPC A61K 8/9722; C12N 1/12; A61Q 19/00

(57) ABSTRACT

The invention relates to a peptide extract of the microalga Chlamydomonas acidophila, and to a method for the preparation thereof. The invention also relates to a composition, advantageously a cosmetic or dermatological composition, comprising such an extract. The invention also relates to such a composition or such an extract for use in the prevention or treatment of disorders or diseases affecting the skin, mucous membranes or skin appendages, for use in the prevention or treatment of vascular disorders, or else for use in the prevention or treatment of adipose tissue modifications. The invention finally relates to a cosmetic care process for the skin, the skin appendages or the mucous membranes, with a view to improving the condition thereof or the appearance thereof, which process consists in administering such a composition or such an extract.

### FIELD OF THE INVENTION

The invention relates to a peptide extract of the microalga Chlamydomonas acidophila and to a cosmetic, dermatological or pharmaceutical composition comprising such an extract. The invention also relates to a process for extracting a peptide extract of Chlamydomonas acidophila, and to the extract obtainable by said process. The invention also relates to a composition or such an extract for use in the prevention or treatment of disorders or pathologies of the skin, mucous membranes or skin appendages, for use in the prevention or treatment of vascular disorders, or for use in the prevention or treatment of alterations of the adipose tissue. Finally, the invention relates to a process for cosmetic care of the skin, mucous membranes or skin appendages, with a view to improving their condition or their appearance, consisting in administering such a composition or such an extract.

### Microalgae

Microalgae are unicellular, eukaryotic organisms which are endowed with photosynthesis and therefore able, like higher plants, to use CO₂ from the air for their metabolism in addition to other nutrients such as phosphorus, nitrates, etc. They were among the first species to colonize the earth. There are about 30 000 described species but there are believed to be many more. Microalgae are found in their natural state, in fresh, brackish and salt water throughout the world.

Microalgae can be cultivated according to processes known to the person skilled in the art, such as in photoreactors in light-, pH- and nutrient-controlled environments, and they have many outlets. They, like higher organisms, are able to synthesize proteins, carbohydrates and lipids. Some lipids are particular, such as complex fatty acids or pigments with particular biological properties (xanthophylls). They have become extremely popular for the possible production of biofuel and their production in bioreactors has expanded. Other outlets are diverse: fish feed (aquarium and fish farms), food and human health (astaxanthin extracted from Haematococcus pluvialis, spirulina proteins) and some outlets in the cosmetic industry.

### PRIOR ART—CHLAMYDOMONAS ACIDOPHILA

Chlamydomonas acidophila of the class Chlorophyceae (Family: Chlamydomonadaceae) is a green freshwater microalga that proliferates in very acidic waters (pH 2.3 to 3.4) and is adapted to environments laden with heavy metals. In particular, it was first identified and collected in volcanic lakes in Argentina. It is said to be rich in phytochelatins, particular structures able to chelate metals, and in carotenoids (beta-carotene, lutein). Apart from the publications concerning its possible culture and development conditions (tolerance to extreme pH and to heavy metals), there is little concerning its composition and use. Its “cousin” Chlamydomonas reinhardtii is used as a model organism in different scientific sectors such as genetics.

### DESCRIPTION OF THE INVENTION

The Applicant has discovered that peptide extracts of the microalga Chlamydomonas acidophila exhibit cosmetic, pharmacological and dermatological properties never described before. In particular, it is the first time that such Chlamydomonas acidophila extracts are used as such, for their specific properties.

The invention relates to a peptide extract of the microalgae Chlamydomonas acidophila.

In the sense of the present invention, “peptide extract” means an extract comprising mainly peptides.

In the sense of the present invention, “peptide” means a polymer of amino acids linked together by peptide bonds. A peptide is characterized in particular by a molecular weight comprised between 200 and 10 000 Daltons (Da), inclusive.

Advantageously, the Chlamydomonas acidophila extract according to the invention comprises at least 20% by weight of peptides, the percentages being expressed with respect to the total weight of said extract. In particular, the extract according to the invention comprises from 20% to 90%, advantageously from 20% to 75%, more advantageously from 30% to 70%, typically 65%, by weight of peptides, the percentages being expressed with respect to the total weight of said extract.

Advantageously, the Chlamydomonas acidophila extract according to the invention is substantially free of any protein, in particular of any residual native protein. Among other things, this avoids allergic reactions and improves the solubility and bioavailability of the extract according to the invention.

In the sense of the present invention, “protein” means biological macromolecules formed of one or more polypeptide chains. Each of these chains consists of a sequence of amino acid residues linked together by peptide bonds. A protein is characterized in particular by a molecular weight greater than 10 000 Daltons (Da).

Advantageously, the Chlamydomonas acidophila extract according to the invention is substantially free of free amino acids. The free amino acids have a molecular weight of less than 200 Da.

In the peptide extract of Chlamydomonas acidophila according to the invention, the peptides advantageously have a molecular weight of less than 3500 Daltons (Da). Advantageously, these peptides cover all the amino acid-based compounds initially present in the extract.

Advantageously, in the extract according to the invention, at least 80%, more advantageously at least 90%, of the peptides have a molecular weight of less than 1000 Da.

Advantageously, in the extract according to the invention, at least 30% of the peptides, more advantageously at least 35%, more advantageously at least 40% of the peptides, have a molecular weight of less than 500 Da.

The molecular weight distribution of the peptides is expressed as a percentage of the total peptide concentration.

In the context of the present invention, the peptide extract of Chlamydomonas acidophila is advantageously obtained by enzymatic hydrolysis, more advantageously in the presence of at least one protease. The extract according to the invention is more advantageously obtainable by the process described below in the description.

The invention also relates to a process for preparing a peptide extract of the microalga Chlamydomonas acidophila, comprising at least one enzymatic hydrolysis step. This step is advantageously carried out under the optimal pH and temperature conditions, known to the skilled person, in particular under the optimal pH and temperature conditions related to the enzyme used.

Advantageously, said enzymatic hydrolysis step is carried out in the presence of at least one protease. Said protease can advantageously be an alkaline protease or an acid protease, advantageously it is an alkaline protease.

Advantageously according to the invention, the process for preparing a peptide extract of Chlamydomonas acidophila comprises at least the following steps:

a) aqueous phase dispersion of the microalga Chlamydomonas acidophila;
b) enzymatic hydrolysis of the aqueous dispersion obtained in step a);
c) heat treatment of the mixture obtained in step b); and
d) recovery of the peptide extract at the end of step c).

In step a), the aqueous phase is advantageously water. Furthermore, the content of the microalgae Chlamydomonas acidophila in the aqueous dispersion is advantageously comprised between 0.1% and 20%, more advantageously 1% and 10%, dry extract equivalent of the microalgae.

The enzymatic treatment (step b) is advantageously carried out by adding at least one protease, advantageously under the optimal pH and temperature conditions known to the skilled person, for example at a pH comprised between 3.0 and 9.0 and typically at a temperature comprised between 20° C. and 90° C. In particular, the enzymatic treatment comprises the addition of an alkaline or acid protease, advantageously an alkaline protease.

The enzymatic hydrolysis step of the process according to the invention is very important, since it transforms or “cuts” the native proteins present in Chlamydomonas acidophila to obtain peptides.

In the context of the present invention, the enzymatic hydrolysis step is advantageously followed by a heat treatment step to denature the enzymes. This heat treatment step is advantageously carried out at a temperature greater than 40° C., typically between 80° C. and 100° C.

In step d), the peptide extract is advantageously recovered by extraction of the dispersion obtained at the end of step c), advantageously with stirring, and advantageously at a pH comprised between 3.0 and 9.0 and at a temperature comprised between 20° C. and 90° C.

Advantageously, the process comprises an additional filtration or centrifugation step, located between steps c) and d), optionally followed by ultrafiltration, diafiltration, or nanofiltration.

The filtration or centrifugation steps, optionally followed by membrane ultrafiltration or diafiltration, are used to remove residual proteins. The nanofiltration steps are used to remove mineral salts or free amino acids, for example.

The process according to the invention advantageously comprises a step of ultrafiltration at 15 kDa, advantageously between 10 and 15 kDa, carried out between steps c) and d), which is used to remove any potentially-allergenic residual protein.

Advantageously, the process according to the invention further comprises a nanofiltration step with, for example, a cut-off threshold comprised between 100 Daltons and 300 Daltons, advantageously between 130 and 300 Daltons, typically between 200 Daltons and 300 Daltons, in order to remove some of the amino acids or mineral salts, following the ultrafiltration step. Advantageously, said nanofiltration step is carried out on a 200 Da membrane.

The aqueous hydrolysate obtained, i.e., the peptide extract according to the invention, can then be physically and microbiologically stabilized by addition of solvent such as glycerol or glycols like 1,3-propanediol in different proportions suitable for such stabilization. Advantageously, glycerol will be present alone or in combination with water or a glycol, advantageously in a proportion comprised between 40% and 95% and preferentially between 50% and 90%, by weight with respect to the total weight of the peptide extract and the solvent. Similarly, glycol and preferentially 1,3-propanediol, will advantageously be present alone or in combination with water or glycerol, advantageously in a proportion comprised between 40% and 95% and preferentially between 50% and 90%, by weight with respect to the total weight of the peptide extract and the solvent. Thus, the present invention further relates to a composition comprising the peptide extract of Chlamydomonas acidophila according to the invention, a solvent selected from glycerol, glycols and mixtures thereof in an effective amount for physical and microbiological stabilizing action, and optionally water. The effective amounts for physical and microbiological stabilizing action are as described above.

There is an alternative in which the peptide extract can be stabilized by drying, by processes known to the skilled person, in the presence or absence of a carrier such as, for example, maltodextrins or acacia fibers (Fibregum® from the company CNI). The carrier content typically varies according to a ratio ranging from 0% to 80% of carrier in relation to the percentage of dry matter obtained in the liquid form of the extract. The extract can be dried by atomization, freeze-drying or any process known to the skilled person and is preferentially dried by freeze-drying to obtain a final powder. The final powder advantageously comprises 30% to 70% by weight of dry matter of the extract, the balance to 100% being the freeze-drying carrier. More advantageously, the final powder comprises 50% dry matter from the extract and 50% freeze-drying carrier, said freeze-drying carrier preferably being of the maltodextrin or acacia fiber type.

Preferentially, by way of example, the peptide extract can be obtained according to the following process:

a) solution of the microalgae Chlamydomonas acidophila in water at a content of about 10% dry extract equivalent of the microalgae;
b) enzymatic hydrolysis by an alkaline protease (Alcalase from the company Lyven);
c) heat treatment to denature the enzymes;
c′) centrifugation, ultrafiltration and diafiltration on 15 kDa membranes in order to eliminate potentially-allergenic residual proteins;
c″) 200 Da membrane nanofiltration to remove mineral salts or free amino acids for example; and
d) recovery of the peptide extract obtained at the end of step c″).

In the context of the process according to the invention, the Chlamydomonas acidophila microalga used as raw material can be derived from a culture in an open environment, for example in “raceways” (oval track-shaped tank used for hatchery rearing), or from a culture in a closed environment, in photobioreactors. Advantageously, said microalgae used as raw material is derived from a culture in a photobioreactor, in particular in a stirred-tank photobioreactor. More advantageously, said microalgae used as raw material is derived from a culture in horizontal tubular wave—ventilated stirred—tank photobioreactors, such as, for example, those developed by the company Microphyt and described in particular in the patent application FR 2 943 685 and the international application WO 2011/058267.

The present invention also relates to a Chlamydomonas acidophila extract obtainable by the above-mentioned process. Such an extract meets the specifications defined above.

The invention also relates to a cosmetic, dermatological or pharmaceutical composition comprising a peptide extract of Chlamydomonas acidophila as active principle and, if need be, a suitable excipient.

Advantageously, in the composition according to the invention, the peptide extract of Chlamydomonas acidophila is as defined above or is obtainable by the above-mentioned process. Thus, said extract is advantageously as defined in the above paragraphs concerning the extract according to the invention as such or those concerning the extract obtainable by the process according to the invention.

Said composition is advantageously formulated to be administered external topically, vaginally or orally.

Advantageously, the composition according to the invention comprises from 0.001% to 10%, advantageously 0.01% to 5%, of said peptide extract of Chlamydomonas acidophila, by weight expressed as dry extract, based on the total weight of the composition.

The composition according to the invention may further comprise one or more other active principles.

According to a first alternative, the various preparations are suitable for topical administration and include in particular creams, emulsions, milks, ointments, lotions, oils, aqueous or hydroalcoholic or glycolic solutions, powders, patches, sprays, shampoos, varnishes or any other product for external application. And according to the following alternatives, the various preparations include in particular intimate hygiene care, oral care, such as for example, toothpastes, oral solutions, gingival gels.

Depending on its nature (cosmetic, pharmaceutical or dermatological), the composition according to the invention may further comprise at least one cosmetically, pharmaceutically or dermatologically acceptable excipient. In particular, the composition according to the present invention may further comprise at least one cosmetically, pharmaceutically or dermatologically acceptable adjuvant known to the skilled person, selected from surfactants, thickeners, preservatives, fragrances, dyes, chemical or mineral filters, moisturizing agents, geothermal waters, etc. The skilled person knows how to adapt the formulation of the composition according to the invention by using his or her general knowledge.

The optimal dosages and galenic forms of the compositions according to the invention can be determined according to the criteria generally taken into account in the establishment of a pharmacological, dermatological or cosmetic treatment adapted to a patient or an animal, such as, for example, the age or body weight of the patient or animal, the severity of his or her general condition, the tolerance to the treatment, the side effects observed, and the skin type.

The invention also relates to an extract according to the invention or an extract obtainable by the process according to the invention or a composition according to the invention, for use in preventing and/or treating:
disorders or pathologies of the skin and/or mucous membranes (for example gums, periodontium, genital mucosa) and/or skin appendages (for example hair and nails);
vascular disorders; and
alterations of the adipose tissue.

The invention also relates to the use of an extract according to the invention or an extract obtainable by the process according to the invention or a composition according to the invention, for the manufacture of a cosmetic, pharmaceutical or dermatological composition for preventing and/or treating:
disorders or pathologies of the skin and/or mucous membranes (for example gums, periodontium, genital mucosa) and/or skin appendages (for example hair and nails);
vascular disorders; and
alterations of the adipose tissue.

The invention further relates to a method for preventing and/or treating:
disorders or pathologies of the skin and/or mucous membranes (for example gums, periodontium, genital mucosa) and/or skin appendages (for example hair and nails);
vascular disorders; and
alterations of the adipose tissue,
   comprising administering, in particular topically, an effective amount of an extract according to the invention or an extract obtainable by the process according to the invention or a composition according to the invention to a subject in need thereof.

In particular, the extract according to the invention or the extract obtainable by the process according to the invention or the composition according to the invention is intended for the prevention and/or treatment of allergic, inflammatory, irritative reactions or pathologies or disorders of the barrier or homeostasis of the skin, immature, normal, or mature/aged skin appendages (hair and nails) and/or mucous membranes (gums, periodontium, genital mucosa).

Advantageously, the composition or extract according to the invention can be used for the prevention and/or treatment of reactions, disorders or pathologies of:

the skin, such as acne, rosacea or erythrocouperosis, psoriasis, vascular disorders, diaper rash, atopic dermatitis, eczema, contact dermatitis, irritant dermatitis, allergic dermatitis, seborrheic dermatitis (cradle cap), psoriasis, sensitive skin, reactive skin, dry skin (xerosis), dehydrated skin, skin with redness, skin erythema, aged or photoaged skin, photosensitized skin, pigmented skin (melasma, post-inflammatory pigmentation, etc.), depigmented skin (vitiligo), skin with cellulite, sagging skin, skin with stretch marks, scabs, chapped skin, punctures, cracks, in particular of the breasts, sunburn, inflammation due to all kinds of rays, irritation by chemical, physical (for example stress for pregnant women), bacteriological, fungal or viral, parasitic (lice, scabies, ringworm, mites, dermatophytes) or radiological agents or by deficiency of innate (antimicrobial peptides) or acquired (cellular, humoral, cytokines) immunity, and/or

the mucous membranes such as gums and periodontium that may present gingivitis (sensitive gums of newborns, hygiene problems, due to smoking or others), periodontal disease, or genital mucosa that may present irritation of the external or internal male or female genital areas and/or

the skin appendages such as immature, normal or mature nails (brittle, fragile nails, etc.) and hair (alopecia, dandruff, hirsutism, seborrheic dermatitis, folliculitis) presenting in particular disorders of the scalp such as androgenetic, acute, localized, scarring, congenital or infant occipital alopecia (or pelade), alopecia areata, chemotherapy/radiotherapy-related alopecia or telogen effluvium, anagen effluvium, pilar dystrophy, trichotillomania, ringworm or greasy or dry dandruff.

The invention also relates to a process for cosmetic care of the skin and/or skin appendages and/or mucous membranes, with a view to improving their condition and/or their appearance, consisting in administering an extract according to the invention or an extract obtainable by the process according to the invention or a composition according to the invention.

In particular, the cosmetic care process firms the skin and reduces the “orange peel” effect advantageously by topical route on the skin and/or skin appendages and/or mucous membranes.

In particular, the invention relates to a process for cosmetic care of the skin and/or skin appendages, to act on the elasticity or firmness of the skin, in particular as a tensor or anti-wrinkle agent, to act on sensitive skin, or to act against pollution, consisting in applying to the skin and/or skin appendages a composition or an extract according to the present invention.

In particular, the invention relates to a process for cosmetic care of the skin and/or skin appendages, with a view to preventing damage to the barrier and dehydration thereof, consisting in applying to the skin and/or skin appendages a composition or an extract according to the present invention.

The invention relates to cosmetic skin care process, with a view to preventing aging, consisting in applying to the skin a composition or an extract according to the present invention.

The composition or extract according to the present invention can also be advantageously used in the prevention and/or treatment of vascular disorders, in particular redness and couperosis.

The composition or extract according to the present invention can also advantageously be used in the prevention and/or treatment of alterations of the adipose tissue. Alterations of the adipose tissue are in particular cellulite or the “orange peel” effect. The composition according to the invention firms up the skin.

The present invention may be illustrated in a non-limiting manner by the following examples.

### DESCRIPTION OF THE FIGURES

FIG. 1 represents the results of the erythema intensity measurements: Active/Placebo/Untreated area comparison. NS: non-significant difference. *: p<0.05 (Example 3B).

FIG. 2 represents the change in blood flow over time (Example 3B).

FIG. 3 represents illustrations of the TEWL results obtained at D0 and D28 (Example 3C).

FIG. 4 represents illustrations of the hydration results obtained at D0 and D28 (Example 3C).

FIG. 5 represents the illustrations of the amount of NMFs quantified at D0 and D28 (Example 3C).

FIG. 6 represents the illustrations of the amount of ceramides quantified at D0 and D28 (Example 3C).

FIG. 7 represents the illustrations of the amount of IL1RA quantified at D0 and D28 (Example 3C).

FIG. 8 represents the illustrations of the Nile red/involucrin ratio at D0 and D28 (Example 3C).

### EXAMPLES

### Example 1: Extract According to the Invention

A peptide extract is obtained according to the following process:

a) solution of the microalga Chlamydomonas acidophila at 10% dry matter in water;
b) hydrolysis by an alkaline protease (Alcalase from the company Lyven);
c) heat treatment at a temperature comprised between 80° C. and 100° C. to denature the enzymes;
c′) centrifugation, ultrafiltration and diafiltration on 15 kDa membranes in order to eliminate potentially-allergenic residual proteins
c″) 200 Da membrane nanofiltration to remove mineral salts or free amino acids or monosaccharides
d) recovery of the peptide extract
e) stabilization in a glycerol/1,3-propanediol mixture

The liquid peptide extract thus obtained has the following characteristics:

### 1—Physicochemical Analysis (%/Total Dry Matter)

Dry extract (2 h, 105° C., ventilated oven): 1.2%
pH: 5.1
α-Amino nitrogen (OPA, leucine equivalent): 29%

### Peptides (Kjeldahl, N×6.25): 70%

### Total ash: 4%

### 2—Profile of Peptide Molecular Weight Distributions

Less than 500 Da: 40%

### Between 500-1000 Da: 55%

### Between 1000-3500 Da: 4%

Greater than 3500 Da: 1%

### Example 2: Tests of Biological Activities of the Extract According to the Invention (In Vitro)

The biological activity of the Chlamydomonas acidophila (CAP) extract obtained in Example 1 was demonstrated in vitro as described below.

These in vitro studies have shown the potential of the CAP extract on:
reinforcement of the skin barrier (in particular the lipid barrier);
skin hydration via hyaluronic acid synthesis, NMF production and osmolyte transport pathways;
antioxidant and anti-inflammatory defenses against a specific stresses or those linked to atmospheric pollution;
an antiaging effect, in particular via the preservation of the homeostasis of the dermal matrix;
the mechanisms of allergy.

I. Preliminary Screening of Activity on Dermal Fibroblasts and Melanized Reconstructed Epidermis

The potential biological activities of the Chlamydomonas acidophila extract were investigated by a gene expression modulation test on dermal fibroblasts and melanized reconstructed epidermis. Thus, the expression of 96 genes of major interest in cutaneous and cosmetic physiology was studied by PCR-array on fibroblasts and melanized reconstructed epidermis.

a. Materials and Methods:

The Chlamydomonas acidophila (CAP) extract at 0.05% dry matter was added to the culture medium of normal human dermal fibroblasts (NHDFs) or melanized reconstructed human epidermis.

After 6 or 24 hours of incubation, the expression of the selected markers was assessed by quantitative RT-PCR (TaqMan microfluidic card). The change in expression of the studied markers compared with the control was expressed as a relative quantity (RQ, RQ>1: increase, RQ<1: decrease).

b. Results:

The most significant results showing the effect of the CAP extract on gene expression in reconstructed epidermis are presented in Table 1 below.

TABLE 1
Variations in the expression of genes of
interest in melanized reconstructed human epidermis.
Relative quantity (RQ) compared with the control =
1/p value determined following a Student’s t-test

| CAP 0.05% MS | QR | p value |
|---|---|---|
| HAS2 Hyaluronan synthase 2 | 2.32 | 0.034 |
| RAB11A Ras-related protein Rab-11A | 2.14 | 0.0387 |
| GBA/GBAP1 Glucosylceramidase | 1.91 | 0.0122 |
| (Beta-glucocerebrosidase) | | |
| SLC6A6 (TAUT transporter) | 1.32 | 0.0181 |
| HAS3 Hyaluronan synthase 3 | 1.23 | 0.0385 |
| PADI1 Peptidyl Arginine Deiminase 1 | 2.23 | >0.05 |
| BLMH Bleomycine Hydrolase | 2.03 | >0.05 |
| CASP14 Caspase 14 | 3.19 | >0.05 |

These results tend to show that the Chlamydomonas acidophila extract, by varying the gene expression of certain markers, could be of particular interest in the following activities:

### Lipid Synthesis and Remodeling in the Epidermal Barrier Function:

The GBA/GBAP1 gene encoding the enzyme glucosylceramidase or β-glucocerebrosidase is overexpressed after 6 h of treatment with CAP.
RAB11A encodes a GTPase (Ras-related protein Rab-11A) involved in the biogenesis of lamellar bodies within keratinocytes. This highlights the importance of RAB11A in the homeostasis of the epidermal barrier.

### Hyaluronic Acid Biosynthesis and Epidermal Hydration:

Hyaluronan synthases-2 and -3 (HAS2 and HAS3) are enzymes responsible for the synthesis of hyaluronic acid (HA).
The gradual expression of SLC6A6 in the spiny and granular layers of the epidermis maintains the necessary hydration in the epidermis in a dry environment.
The PADI1, BLMH and CASP14 genes code for enzymes involved in the production of natural moisturizing factors (NMFs).

Table 2 below presents the most significant results of the CAP extract on gene expression in fibroblasts.

TABLE 2
Variations in the expression of genes of
interest in normal human dermal fibroblasts (NHDFs).
Relative quantity (RQ) compared with the control equal
to 1/p value determined following a Student’s t-test

| CAP 0.05% ms | QR | p value |
|---|---|---|
| HAS 2 Hyaluronan synthase 2 | 5.76 | 0.0082 |
| NQO1 NAD(H)dehydrogenase, quinone 1 | 2.81 | 0 |
| MKI67 Antigen Ki-67 | 2.79 | 0.0005 |
| PRDX6 Peroxiredoxin-6 | 1.98 | 0.0059 |
| TXNRD1 Thioredoxin reductase 1 | 1.75 | 0.0007 |
| LMNB1 Lamin-B1 | 3.25 | 0.0003 |
| PSMD1 26S proteasome non-ATPase | 1.72 | 0.0072 |
| regulatory subunit 1 | | |
| HSPA1A Heat shock 70 kDa protein 1A | 1.57 | 0.0063 |
| LOXL2 Lysyl oxidase homolog 2 | 1.40 | 0.0484 |
| SLC2A1/GLUT-1 Solute carrier family 2, | 1.38 | 0.0291 |
| facilitated glucose transporter member 1 | | |
| GLO1 Lactoylglutathione lyase (glyoxalase 1) | 1.19 | 0.0444 |

These results show a potential activity of the CAP extract in the following areas:

### Skin Anti-Aging:

The three hyaluronan synthases, HAS1, HAS2 (5.76 0.008) and HAS3, produced by fibroblasts in the dermis are responsible for the biosynthesis of hyaluronic acid (HA).
Furthermore, it has been shown that skin aging is associated with a decrease in fibroblast proliferation. A decrease in the expression of the proliferation factor Ki-67 is observed in relation to age [Ma et al. 2011 Br. J. Dermatol, 164(3), pp. 479-482]. The CAP active agent increases the expression of MKI67 after 24 h of treatment, testifying to a possible increase in cell proliferation, which reinforces its anti-aging effect.
It has been shown that the expression of lamin B1 (LMNB1) in the skin decreases with age.

### Antioxidant Defenses:

NAD(P)H dehydrogenase, quinone 1 (NQO1) is a cytosolic flavoprotein under the control of the transcription factor Nrf-2. NQO1 promotes, by reduction, the formation of hydroquinones from quinones, preventing the production of radical species.
The HSP70 protein (encoded by the HSPA1A gene) is a chaperone molecule (heat shock protein 70) that inhibits the aggregation of denatured proteins, promotes their renaturation (refolding) and controls essential mediators of the apoptotic machinery.
The thioredoxin system is one of the major antioxidant defense systems. Among the 3 isoforms of thioredoxin reductase, the isoform 1 encoded by the TXNRD1 gene regulated by the CAP extract is the most studied.
Besides the thioredoxin system, the peroxiredoxin/sulfiredoxin system is also present. Among the peroxiredoxins, peroxiredoxin-6 (PRDX6) is overexpressed by the CAP extract after 24 h of treatment. This indicates a detoxifying action of the active agent towards the presence of peroxides.
Finally, the CAP extract stimulates the GLO1 gene, which is part of the GLO system that detects and neutralizes certain carbonyls, thus preventing them from attacking cells and their components.

### Dermal Matrix Homeostasis:

The CAP extract stimulates the expression of PSMB1, the gene encoding the beta 1 subunit of the proteasome. The proteasome plays a major role in maintaining protein homeostasis by removing damaged or malformed proteins that could alter cellular function.
The gene expression of LOXL2 is also increased, this gene is part of the lysyl oxidase family, enzymes involved in the assembly of elastin and collagen fibers.

II. Anti-Inflammatory Action

### A. Anti-Inflammatory Activity Against PMA Chemical Stress

a. Introduction:

The inflammatory response is the normal, immediate and transient response of the body to any environmental attack.

However, in certain pathological or physiological conditions, this inflammatory reaction can be exacerbated and, if not properly controlled, can lead to tissue damage.

In the skin, the keratinocyte is one of the first cells involved in the initiation of the inflammatory reaction in response to environmental attack.

The “attacked” keratinocyte will then release:
primary cytokines (IL1α, IL1β or TNFα) or secondary cytokines (IL8) that will induce a cascade of reactions involving the immune system.
prostaglandins (PGEs), which are members of the prostanoid family. The prostaglandin synthesis pathway that leads to the synthesis of PGE2 and other PGEs is induced by inflammatory stimuli.

The anti-inflammatory activity of the Chlamydomonas acidophila extract according to the invention was evaluated on a model of inflammation induced on keratinocytes by PMA (phorbol 12-myristate 13-acetate) treatment. The release of the cytokines TNFα and prostaglandin E2 (PGE2) was analyzed.

b. Materials and Methods:

Normal human epidermal keratinocytes were pretreated for 24 h with the Chlamydomonas acidophila (CAP) extract according to Example 1, at concentrations comprised between 0.0001% and 0.05% dry matter, or with the anti-inflammatory reference molecules dexamethasone at 0.1 μM or indomethacin at 0.1 μM (the latter two references serving as anti-inflammatory references for cytokines and prostaglandins, respectively).

Inflammation was then induced by addition of PMA at 10 μg/mL overnight.

A TNFα and PGE2 assay was then performed in the cell culture supernatants.

The significance of the results was checked by a one-way ANOVA followed by a Tuckey test (GraphPad Prism software version 5.02, GraphPad Software, San Diego Calif. USA).

c. Results:

PMA at 10 μg/ml significantly increased the release of TNFα in keratinocyte supernatants and thus did induce inflammation. Dexamethasone at 0.1 μM and indomethacin at 0.1 μM, as a 24 h pretreatment, did decrease TNFα release, demonstrating their anti-inflammatory effect and validating the test.

The Chlamydomonas acidophila extract, as a 24 h pretreatment at different concentrations, significantly decreased TNFα release and thus showed anti-inflammatory action against PMA.

TABLE 3
TNF-alpha assay in normal human keratinocytes stimulated with PMA
$$$ p <0.001 vs Control/*** p <0.001 vs PMA-One-way
ANOVA followed by a Tukey test

| | TNF-alpha (pg/ml) | | | |
|---|---|---|---|---|
| | | Standard | | |
| | Mean | deviation | Change (%) | Significance |
| Control | 22.683 | 9.077 | | |
| 10 μg/ml PMA | 286.562 | 74.204 | +1163% vs Ctrl | $$$ |
| 0.1 μM dexamethasone + PMA | 126.112 | 31.618 | −56% vs PMA | ** |
| 0.1 μM indomethacin + PMA | 113.266 | 22.351 | −60% | *** |
| 0.0001% CAP + PMA | 70.599 | 5.007 | −75% | *** |
| 0.001% CAP + PMA | 61.262 | 11.948 | −79% | *** |
| 0.01% CAP + PMA | 17.417 | 1.827 | −94% | *** |
| 0.05% CAP + PMA | 57.256 | 7.640 | −80% | *** |

PMA at 10 μg/ml significantly increased the release of PGE2 in keratinocyte supernatants and thus PMA did induce inflammation. Indomethacin and dexamethasone at 0.1 μM, as a 24 h pretreatment, significantly decreased the release of PGE2. The anti-inflammatory effect of these two references was thus well validated. The Chlamydomonas acidophila extract, as a 24 h pretreatment at both concentrations, significantly decreased the release of PGE2 and thus showed an anti-inflammatory action against PMA.

TABLE 4
PGE2 assay in normal human keratinocytes stimulated with PMA
$$$ p <0.001 vs control/** p <0.01; *** p <0.001 vs PMA-One-way
ANOVA followed by a Tukey test

| | PGE2 (pg/ml) | | | |
|---|---|---|---|---|
| | | Standard | | |
| | Mean | deviation | Change (%) | Significance |
| Control | 217.444 | 33.066 | | |
| 10 μg/ml PMA | 1255.398 | 68.537 | +477% vs Ctrl | $$$ |
| 0.1 μM dexamethasone + PMA | 985.126 | 98.254 | −22% vs PMA | ** |
| 0.1 μM indomethacin + PMA | 163.031 | 49.773 | −87% vs PMA | *** |
| 0.0001% CAP + PMA | 355.958 | 114.148 | −72% vs PMA | *** |
| 0.001% CAP + PMA | 423.269 | 29.429 | −66% vs PMA | *** |
| 0.01% CAP + PMA | 318.410 | 96.675 | −75% vs PMA | *** |
| 0.05% CAP + PMA | 487.345 | 38.567 | −61% vs PMA | *** |

d. Conclusion:

The anti-inflammatory effect of the Chlamydomonas acidophila extract was demonstrated through its action on the release of TNFα and prostaglandin E2 under inflammatory conditions.

### B. Anti-Inflammatory Activity Against Nickel Stress

a. Introduction

Nickel is the major cause of allergic contact dermatitis in the population, with a worldwide prevalence of roughly 8.6%. The objective of the study described below is to evaluate the effect of the CAP extract on the release of IL8 by nickel-stimulated keratinocytes.

b. Materials and Methods

Normal human epidermal keratinocytes were pretreated for 24 hours with CAP extract at 0.01% and 0.05% dry matter or with the anti-inflammatory reference molecule dexamethasone at 1 μM. The keratinocytes were then treated for 24 hours with nickel: NiSO₄ at 10 μM. At the end of the incubation, the amount of IL8 produced by the cells was assessed by ELISA in the supernatants.

The concentration of IL8 assayed was normalized to the amount of total intracellular protein assessed by BC Assay.

Significance of results was statistically analyzed by a Student's t-test.

c. Results

The CAP extract induces a significant decrease in the release of IL8 induced by nickel stress in keratinocytes.

TABLE 5
IL8 assay in NiSO₄-stimulated normal human keratinocytes

| | IL8 (pg/ml/mg protein) | | | |
|---|---|---|---|---|
| | | Standard | | |
| | Mean | deviation | Change (%) | t test |
| Control | 483 | 545 | | |
| 10 μM NiSO₄ | 4596 | 1161 | +852% vs Ctrl | p <0.001 |
| 1 μM dexamethasone + NiSO₄ | 1685 | 871 | −63% vs Ni | p <0.01 |
| 0.01% CAP + NiSO₄ | 1608 | 870 | −65% vs Ni | p <0.05 |
| 0.05% CAP + NiSO₄ | 2303 | 914 | −50% vs Ni | p <0.01 |

d. Conclusion

The Chlamydomonas acidophila (CAP) extract inhibits the release of a major cytokine, IL8, in the context of nickel-induced inflammatory stress. The extract is therefore of interest in the context of contact allergy or cutaneous hypersensitivity related to nickel.

### C. Anti-Inflammatory Activity Against Cadmium Stress

a. Introduction

The objective of this study is to evaluate the anti-inflammatory activity of the Chlamydomonas acidophila (CAP) extract against heavy metal stress, represented by cadmium, on normal human keratinocytes.

b. Materials and Methods

Normal human epidermal keratinocytes were pretreated for 24 hours with the CAP extract at 0.001% and 0.01% dry matter or with the anti-inflammatory reference molecule indomethacin at 0.1 M. The keratinocytes were then treated for 48 hours with cadmium: CdCl₂ at 100 μM. At the end of the incubation, the amount of PGE2 produced by the cells was assessed by ELISA in the supernatants.

The concentration of PGE2 assayed was normalized to the amount of total intracellular protein assessed by BC Assay.

c. Results

The CAP extract induces a decrease in the release of PGE2 induced by cadmium stress in keratinocytes.

TABLE 6
PGE2 assay in cadmium-stimulated normal human keratinocytes

| | PGE2 (pg/μg of protein) | | |
|---|---|---|---|
| | | Standard | |
| | Mean | deviation | Change (%) |
| Control | 0.814 | 0.161 | |
| 100 μM CdCl₂ | 8.338 | 3.036 | +925% vs Ctrl |
| 0.1 μM indomethacin + CdCl₂ | 3.874 | 1.825 | −54% vs Cd |
| 0.001% CAP + CdCl₂ | 7.377 | 2.827 | −12% vs Cd |
| 0.01% CAP + CdCl₂ | 5.810 | 0.517 | −30% vs Cd |

d. Conclusion

The Chlamydomonas acidophila (CAP) extract inhibits the production of prostaglandin E2 (PGE2) induced by cadmium stress. The extract thus provides a protection of the skin toward heavy metal stress, in the context of environmental pollution, for example.

### D. Anti-Inflammatory Activity Against SDS Stress

a. Introduction

The anti-inflammatory activity of the Chlamydomonas acidophila (CAP) extract was assessed on a model of inflammation induced by sodium dodecyl sulfate (SDS) treatment on reconstructed epidermis.

b. Materials and Methods

Reconstructed human epidermises (RHE) were preincubated for 24 hours in the presence of CAP at 0.01% and 0.05% dry matter. SDS at 0.025% was then applied to the surface of the epidermises which were again incubated for 24 hours, again in the presence of the CAP extract.

At the end of incubation, the cytokine tumor necrosis factor alpha (TNFα) was assayed by ELISA in the supernatants.

Gene expression of inflammatory and barrier markers was assessed by qRT-PCR.

Significance of results was statistically analyzed by a one-way ANOVA followed by a Tukey test.

c. Results

SDS treatment of reconstructed epidermis induces an increase in TNFα expression at the gene level (qRT-PCR, Table 8) and protein level (ELISA, Table 7). This proinflammatory effect is also accompanied by a decrease in keratin 1 (KRT1) expression (Table 8), testifying to an impairment of epidermal barrier function.

Under these conditions, the CAP extract significantly inhibited TNFα overproduction and increased keratin-1 expression.

TABLE 7
Assay of TNFα produced by SDS-
stimulated reconstructed human epidermises.

| | TNFα (pg/ml) | | | |
|---|---|---|---|---|
| | | Standard | | |
| | Mean | deviation | Change (%) | Significance |
| Control | 4.469 | 0.202 | | |
| 0.025% SDS | 23.019 | 4.426 | +415% vs Ctrl | p <0.001 |
| 0.01% CAP + SDS | 14.923 | 2.456 | −35% vs SDS | p <0.05 |
| 0.05% CAP + SDS | 12.213 | 2.751 | −47% vs SDS | p <0.01 |

TABLE 8
Gene expression in SDS-stimulated reconstructed human epidermises
* p <0.05; *** p <0.001-One-way ANOVA followed by a Tukey test

| | KRT1 | | TNFα | |
|---|---|---|---|---|
| | Mean relative | | Mean relative | |
| | quantity | Change (%) | quantity | Change (%) |
| Control | 0.96 | | 1.4 | |
| 0.025% SDS | 0.46 | −52% vs Ctrl | 18.5 | +1235% vs Ctrl *** |
| 0.01% CAP + SDS | 0.67 | +44% vs SDS | 15.9 | −14% vs SDS |
| 0.05% CAP + SDS | 0.53 | +14% vs SDS | 9.7 | −48% vs SDS * |

d. Conclusion

These results confirm the anti-inflammatory potential of the Chlamydomonas acidophila extract and show its ability to protect the barrier from external stress.

III. Antioxidant Action

a. Introduction

The gene expression screening performed on the Chlamydomonas acidophila extract according to Example 1 having shown a potential in the stimulation of antioxidant defenses; the capacity to protect the cell from an oxidative stress was evaluated by measuring the production of reactive oxygen species (ROS) in keratinocytes submitted to oxidative stress induced by H₂O₂.

The evaluation of the antioxidant effect of the active agent is done through the incorporation of DCFH-DA (2′,7′-dichlorofluorescin diacetate) into cultured keratinocytes. This molecule is a non-fluorescent marker in the non-oxidized state. Under oxidizing conditions (here H₂O₂ stress), DCFH-DA will be degraded to DCF, a molecule that will emit fluorescence. The fluorescence measured will be proportional to the amount of reactive oxygen species produced by the cell in the presence of H₂O₂ and/or the extract.

b. Materials and Methods

Normal human epidermal keratinocytes were preincubated for 24 hours in the presence of the CAP extract at 0.0001% dry matter, quercetin at 10 μM or vitamin C at 500 μM (the latter two molecules serving as antioxidant reference).

The cells are then treated for 1 h in the presence of 0.5 mM DCFH-DA.

Oxidation is induced by adding 100 μM H₂O₂ for 20 minutes. A second treatment with the tested products is performed simultaneously with H₂O₂ stress (at the same concentrations as the pretreatment).

Finally, a measurement of the fluorescence density (DFU) corresponding to the amount of ROS is performed using a microplate reader.

The significance of the results was checked by a one-way ANOVA followed by a Tuckey test (GraphPad Prism software version 5.02, GraphPad Software, San Diego Calif. USA).

c. Results

An increase in ROS production was observed after H₂O₂ treatment, validating the model. Quercetin and vitamin C significantly decreased ROS production after H₂O₂ treatment. The antioxidant effect of these two references was well validated on the model.

The Chlamydomonas acidophila extract significantly decreased the production of ROS induced by H₂O₂ stress.

TABLE 9
Production of reactive oxygen species (ROS) in
keratinocytes treated with hydrogen peroxide (H₂O₂)
*** p <0.001-One-way ANOVA followed by Tuckey test

| | ROS (fluorescence units) | | | |
|---|---|---|---|---|
| | | Standard | | |
| | Mean | deviation | Change (%) | Significance |
| Control | 10904.67 | 1331.27 | | |
| 100 μM H₂O₂ | 102854.67 | 7572.06 | +843% vs Ctrl | *** |
| Quercetin + H₂O₂ | 7787.00 | 1063.33 | −92% vs H₂O₂ | *** |
| Vitamin C + H₂O₂ | 10932.50 | 1131.55 | −89% vs H₂O₂ | *** |
| 0.0001% CAP + H₂O₂ | 72375.67 | 9318.04 | −30% vs H₂O₂ | *** |

d. Conclusion:

The Chlamydomonas acidophila extract has demonstrated an antioxidant effect against H₂O₂-induced stress.

IV. Activity on the Barrier and Hydration

a. Introduction

The gene expression screening performed on the Chlamydomonas acidophila extract and presented above showed a potential effect on the stimulation of the expression of gene markers involved in the barrier and hydration. We sought to confirm this effect on keratinocytes.

b. Materials and Methods

Normal human epidermal keratinocytes were incubated for 48 hours in the presence of the CAP extract at 0.001% dry matter.

Gene expression of barrier function and hydration markers was assessed by qRT-PCR.

The results were statistically analyzed by a one-way ANOVA followed by a Dunnett's test.

c. Results

The Chlamydomonas acidophila extract stimulated the gene expression of the markers GBA (beta-glucocerebrosidase) and HAS3 (hyaluronan synthase-3) involved in the synthesis of epidermal lipids and hyaluronic acid, respectively. These results, in favor of an effect of reinforcement of the epidermal permeable barrier and hydration, confirm the trends observed in the context of the genomic expression screening.

Furthermore, the Chlamydomonas acidophila extract also stimulated the expression of the markers SLC6A6 and SLC5A3, encoding TAUT (taurine membrane transporter channel) and SMIT (myoinositol transporter channel), respectively.

These two genes code for osmolyte transporters and are therefore involved in maintaining skin hydration and protecting cells from external stresses.

Finally, the extract induced an increase in the gene expression of filaggrin (FLG) and PADI1 (peptidyl arginine deiminase), protein and enzyme involved in the synthesis of natural moisturizing factor (NMF) elements.

TABLE 10
Gene expression of barrier and hydration markers in keratinocytes
* p <0.05-One-way ANOVA followed by Dunnett’s test

| | | Control | 0.001% CAP |
|---|---|---|---|
| HAS3 | Relative Quantity | 0.77 | 1.2 |
| | Change vs Ctrl (%) | | +56% |
| GBA | Relative Quantity | 1.05 | 3.33 |
| | Change vs Ctrl (%) | | +217% * |
| SLC6A6 | Relative Quantity | 1.16 | 1.8 |
| | Change vs Ctrl (%) | | +56% |
| SMIT | Relative Quantity | 1.06 | 1.82 |
| | Change vs Ctrl (%) | | +71% |
| FIG | Relative Quantity | 0.95 | 1.41 |
| | Change vs Ctrl (%) | | +48% |
| PADI1 | Relative Quantity | 1.11 | 1.94 |
| | Change vs Ctrl (%) | | +74% * |

d. Conclusion

These results show that the extract of Chlamydomonas acidophila has a potential in the reinforcement of the skin barrier and the maintenance of skin hydration.

V. Evaluation of the Effects of the Chlamydomonas acidophila Extract in the Mechanisms of Allergy

### A. Introduction

The potential anti-allergic effects of the Chlamydomonas acidophila extract were investigated on:
Gene expression of pro-inflammatory chemokines in normal human epidermal keratinocytes (NHEK) stimulated by a mixture of Th2 cytokines (IL-4+IL-13+IL-22+TNF-α) mimicking a late-phase “atopic dermatitis” type phenotype (chronic inflammation).
Activation of human basophils induced by fMLP. This activation was measured using a specific kit and flow cytometry analysis by quantifying a specific marker of activated basophil cells (CD63) in the total population of basophils identified by expression of the CCR3 marker. In parallel, activation with the anti-FCεRI antibody was performed as a positive control.

### B. Inhibition of Chemoattractant Factors Following TH2 Stress

a. Materials and Methods

Normal human epidermal keratinocytes were preincubated for 24 hours in the presence of the CAP extract at 0.01% dry matter (DM) or reference JAK inhibitor I at 10 μM. After preincubation, the cells were retreated with the CAP extract or reference and then the cells were stimulated with a Th2 cytokine cocktail (IL4+1L13+1L22+TNFα at 10 ng/ml) for 24 hours.

At the end of incubation, gene expression of markers of interest was assessed by qRT-PCR.

b. Results

In keratinocytes subjected to Th2 stress, the Chlamydomonas acidophila extract inhibited the gene expression of CCL5 (C-C motif chemokine ligand 5 or RANTES) and CCL27 (C-C motif chemokine ligand 27), encoding chemokines involved in the amplification of the cutaneous inflammatory and allergic response.

TABLE 11
Gene expression in epidermal keratinocytes subjected to Th2 stress

| | CCL5 | | CCL27 | |
|---|---|---|---|---|
| | Relative | Change | Relative | Change |
| | expression (%) | (%) | expression (%) | (%) |
| Control | 100 | 100 | | |
| Th2 stress | 345 | +245% vs Ctrl | 848 | +748% vs Ctrl |
| JAK inhibitor + Th2 | 101 | −7/% vs Th2 | 1436 | / |
| 0.01% CAP + Th2 | 176 | −49% vs Th2 | 502 | −41% vs Th2 |

c. Conclusion

The Chlamydomonas acidophila extract modulates Th2 stress-induced inflammation in keratinocytes by inhibiting gene expression of the chemoattractant factors CCL5 and CCL27.

### C. Inhibition of Basophil Activation

a. Materials and Methods

The basophil activation test (BAT) was performed using the Flow CAST® kit (BUHLMANN, item code FKCCR).

Whole blood was preincubated for 15 minutes in the presence of the CAP extract at 0.033% and 0.1% dry matter (DM) or the references (SB202190 at 30 μM; or cromoglycate at 10 mM).

The stimulant, 1 μM fMLP, was then added and the blood was incubated for an additional 15 minutes in the presence of the labeling buffer containing a mixture of monoclonal antibodies (anti-CD63-FITC and anti-CCR3-PE).

Flow cytometry analysis was then performed to count the total population of basophils (CCR3+) and activated basophils (CCR3+/CD63+).

b. Results

Stimulation with the fMLP peptide resulted in a very clear activation of basophils (40.1% activated cells, or 4527% stimulation).

In this study, 2 potential reference compounds were tested in the presence of fMLP:
SB202190, an inhibitor of p38 MAP kinase; the activation of this kinase is essential in the activation mechanism of basophils leading to degranulation;
Cromoglycate, a known anti-allergic, whose mechanism of action involves stabilization of the plasma membrane at which it inhibits the intracellular penetration of Ca⁺⁺, this ion being essential for mast cell degranulation.

Both SB202190, tested at 30 μM, and cromoglycate, tested at 10 mM, showed a significant inhibitory effect on fMLP-induced basophil activation (26% and 46% inhibition, respectively).

Under the experimental conditions of this study, the CAP extract, tested at 0.033% and 0.1%, showed a clear concentration-dependent inhibitory effect on fMLP-induced basophil activation (22% and 39% inhibition, respectively).

TABLE 12
Effect of compounds on the activation of
human basophils under ƒMLP-stimulated conditions
Flow cytometry analysis after double labelling with
anti-CCR3 and anti-CD63 Student’s t-test

| | | % of activated basophils | | |
|---|---|---|---|---|
| | | (CCR3+/CD63+) | | Inhibition vs |
| | | Mean | SEM | ƒMLP (%) |
| Unstimulated | Control | 0.87 | 0.1 | |
| condition | | | | |
| 1 μM fMLP | 1 μM fMLP | 40.5 | 2.5 | |
| stimulated | 30 μM SB202190 | 30.4 | 0.6 | −26% * |
| conditions | 10 μM cromoglycate | 22.3 | 1.3 | −46% ** |
| | 0.033% DM CAP | 31.9 | 1.4 | −22% * |
| | 0.1% DM CAP | 25.1 | 1 | −39% ** |

c. Conclusion

The Chlamydomonas acidophila extract inhibits basophil activation.

### D. Conclusion

By inhibiting, on the one hand, the gene expression of chemoattractant cytokines in keratinocytes subjected to Th2 stress, and on the other hand, the activation of basophils; the Chlamydomonas acidophila extract could contribute to modulate the processes involved in the initiation of the allergic response.

### Example 3: Clinical Tests of the Extract According to the Invention

The biological activity of the Chlamydomonas acidophila (CAP) extract obtained in Example 1 was demonstrated by clinical studies as described below.

All of these results demonstrate significant effects of the “CAP” active agent, such as:
a protective effect;
a barrier effect;
a moisturizing effect; and
an anti-inflammatory/anti-redness effect.

These clinical studies have highlighted the potential of the CAP extract for the prevention or treatment of:
Sensitive skin;
Redness;
Inflammation; and
Allergies.

### A. Summary of Clinical Results

### Chemical Erythema Study

The CAP active agent (3% active matter) has demonstrated significant efficacy on the following parameters:

Intensity of Maximum Erythema 20 Minutes after Application of a 0.1% Methyl-Nicotinate Solution

The blood flow intensity measured by TiVi is significantly lower in the area treated with the active agent compared with the untreated area.

The redness measured by spectrocolorimetry is significantly lower on the area treated with the active agent compared with the untreated area.

Change in the Erythema 30 Minutes after the Maximum Intensity Erythema

The decrease in blood flow intensity measured by TiVi is significantly higher in the area treated with the active agent compared with the untreated area.

The reduction in redness measured by spectrocolorimetry is significantly greater in the area treated with the active agent compared with the untreated area.

### Sensitive Skin Study

The CAP active agent (3% active matter) has demonstrated significant efficacy on the following parameters:

Instrumental Measurements

The active agent:
significantly reduces transepidermal water loss after 28 days of application.
significantly reduces redness after 28 days of application.
significantly increases hydration after 28 days of application.

Biochemical Assessments

The active agent:
significantly increases the amount of NMFs after 28 days of application.
significantly compensates for the decrease in ceramides observed in the placebo area after 28 days of application.
significantly reduces the amount of IL1RA after 28 days of application.
significantly decreases the amount of IL1α after 28 days of application.
significantly decreases the IL1RA/IL1α ratio after 28 days of application.
significantly reduces the amount of IL8 after 28 days of application.

### B. Evaluation of the Protective/Anti-Redness/Anti-Inflammation Efficacy of an Active Agent Versus Placebo by Evaluation of Blood Flow by TiVi and Evaluation of Color by Spectrocolorimeter

### Study Design:

Double-blind study.
Randomized, active agent vs placebo comparative study

### Population:

Nineteen (19) subjects were analyzed for this study. The 19 subjects applied the active agent and the placebo on 2 defined areas. An untreated area was also defined.

The individuals recruited for this study were:
healthy females between 18 and 60 years old, mean age 38±3, min: 18, max: 60
with Caucasian, phototype I to III skin

### Methods of Use and Conduct of the Study:

Application of the products by the subjects themselves, at home, twice a day (morning and evening) for 14 days from D−14 to D0t0 on each defined area of the forearm. After 14 days, subjects returned to the clinical unit. Basal D0t0 measurements are then taken. A final application of the products is performed. A chemical erythema is then induced on each area with a 0.1% methyl-nicotinate solution. Measurements on each area are then taken after 20 minutes (maximum intensity) denoted D0t20 and 50 minutes (30 minutes after the maximum erythema) denoted D0t50 following the induction of erythema.

Two parameters are analyzed for each parameter evaluated:
The variation between D0t20 and D0t0 reflecting the preventive effect on the appearance of erythema of each product.
The variation between D0t50 and D0t20 reflecting the preventive effect on the change in erythema of each product.

### Evaluation of Blood Flow by Tivi

The method used by the TiVi 700 is based on the fact that green light is strongly absorbed by blood vessel cells, while red light is moderately absorbed. By using a polarized light source, the method does not take into account the specular reflection but only the light reflected by the skin tissue. The device produces an intensity map with each pixel representing a concentration of blood cells in the skin.
A decrease in the intensity of the concentration of blood cells in the skin reflects an anti-inflammatory effect.

### Results: Preventive Effect on the Appearance of Erythema

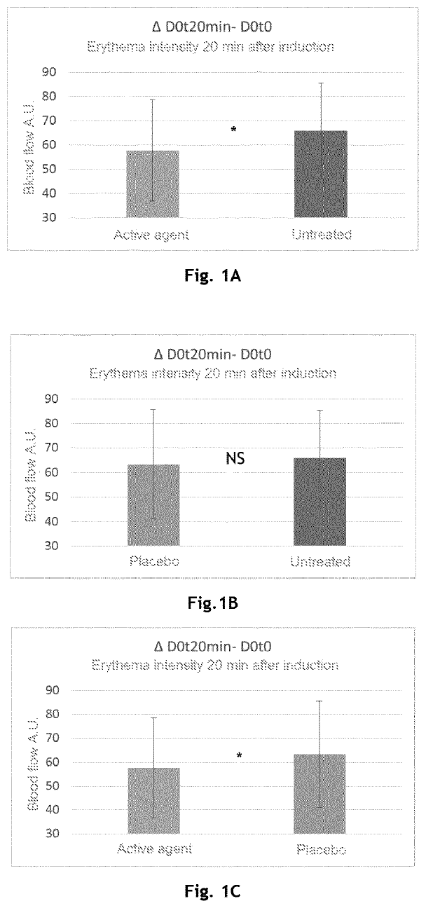

The graphs in FIGS. 1A, 1B, and 1C represent pairwise comparisons between the active agent, the placebo, and the untreated area on the intensity of erythema 20 minutes after methyl-nicotinate application measured by TiVi. The ordinate parameter represents the intensity of blood flow (red blood cell concentration). The exact numerical values are in Tables 13A and 13B below (Tables 13A and B: mean and standard deviation of measurements with % of subjects exhibiting a positive effect. % difference and p value (exact value of significance).

The graph in FIG. 2 shows the change in blood flow values over time.
The results show that the active agent significantly decreases the intensity of the erythema created compared with the untreated area. The intensity of the erythema on the placebo area did not differ from the treated area. The intensity of the erythema on the active area is significantly lower compared with the placebo.

TABLE 13A

| | Δ D0t20 min-D0t0 | | |
|---|---|---|---|
| | | Standard | % |
| Area | Mean | deviation | positive |
| Active agent | 57.6 | 20.9 | 79% |
| Placebo | 63.3 | 22.2 | 68% |
| Untreated | 65.7 | 19.7 | |

TABLE 13B

| | Δ D0t20 min-D0t0 | |
|---|---|---|
| | % diff. | p value |
| Active agent vs Untreated | −12.3 | 0.010 |
| Placebo vs Untreated | −3.8 | 0.349 |
| Active agent vs Placebo | −9.8 | 0.024 |

### C. Evaluation of the Efficacy of an Active Agent Versus Placebo by Evaluation of Transepidermal Water Loss by Tewameter, Evaluation of Hydration by Corneometry, Evaluation of Biochemical Properties by Swabbing

### Study Design:

Double-blind study.
Active agent vs placebo, randomized, half-face comparative study

### Population:

Regarding the measurement of hydration, transepidermal water loss, color and questionnaire, 36 subjects were included in the analysis.

Regarding biochemical analyses, 10 subjects were included in the analysis.

The individuals recruited for this study were:
healthy females, aged over 18, mean age 51±3, min: 21, max: 68
with Caucasian skin, phototype I to IV
having sensitive skin on the face (the subject's skin must react to at least 2 of the following 3 stresses: environmental, chemical or mechanical)

### Methods of Use and Study Conduct:

At D0:
Verification of inclusion and non-inclusion criteria
Acclimatization for 30 minutes
Definition of the measurement areas on each half-face
Instrumental measurements and biological samples

Between D0 and D28:
Half-face application of the active agent and the placebo twice a day (morning and evening)

At D28:
Acclimatization for 30 minutes
Instrumental measurements and biological samples

### Evaluation of Transepidermal Water Loss

The skin barrier regulates water loss through evaporation. When this barrier is damaged, transepidermal water loss increases. Conversely, a reinforced barrier corresponds to lower transepidermal water loss.

Transepidermal water loss was measured by a Tewameter TM 300. The principle is to measure the temperature and relative humidity in a tube with one of its openings applied to the skin by 2 sensors located at 2 different heights. Fick's law is then used to determine transepidermal water loss.

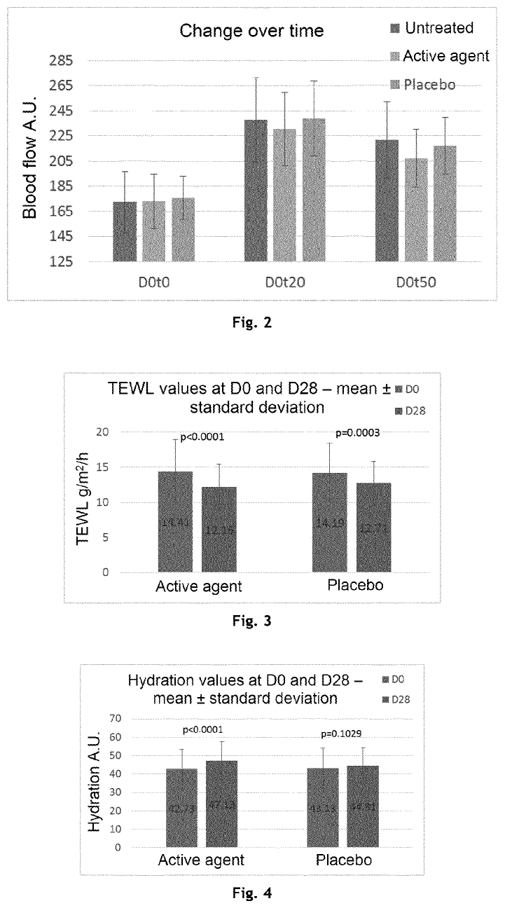

The results obtained for the TEWL measurement at D0 and D28 are given in Table 14. The illustration of the changes is given in FIG. 3.

TABLE 14
Transepidermal water loss values at D 0 and D 28. Percentage changes and statistics.

| | | | | | | | | p value | % Active |
|---|---|---|---|---|---|---|---|---|---|
| | | Active agent | | | | | | Active agent | agent vs |
| TEWL | | D 0 | D 28 | ΔD 28-D 0 | % D 28-D 0 | % respondents | p value D 0 vs D 28 | vs Placebo | Placebo |
| | Mean | 14.41 | 12.16 | −2.25 | −15.63 | 72% | <0.0001 | 0.0216 | −52.7 |
| | SD | 4.50 | 3.26 | | | | | | |
| | | Placebo | | | | | | | |
| | | D 0 | D 28 | ΔD 28-D 0 | % D 28-D 0 | % respondents | p value D 0 vs D 28 | | |
| | Mean | 14.19 | 12.71 | −1.48 | −10.40 | 50% | 0.0003 | | |
| | SD | 4.26 | 3.12 | | | | | | |

The results obtained show that TEWL decreases significantly between D0 and D28 with the active agent and the placebo. The comparison of the change between D0 and D28 observed with the active agent versus the change observed with the placebo is statistically significant in favor of the active agent.

### Evaluation of Hydration

The measurement of skin hydration was performed by a CM 825 Corneometer. This device is based on the principle of capacitance measurement, allowing a measurement of the hydration of the superficial layers of the skin (10 to 20 μm deep).

The results obtained for hydration at D0 and D28 are given in Table 15. The illustration of the changes is given in FIG. 4.

The results obtained show that hydration increases significantly between D0 and D28 with the active agent while it does not vary with the placebo. The comparison of the change between D0 and D28 observed with the active agent versus the change observed with the placebo is statistically significant in favor of the active agent.

TABLE 15
Values at D 0 and D 28 of hydration. Percentage changes and statistics.

| | | | | | | | | p value | % Active |
|---|---|---|---|---|---|---|---|---|---|
| | | Active agent | | | | | | Active agent | agent vs |
| Hydration | | D 0 | D 28 | ΔD 28-D 0 | % D 28-D 0 | % respondents | p value D 0 vs D 28 | vs Placebo | Placebo |
| | Mean | 42.73 | 47.13 | 4.40 | 10.31 | 75% | <0.0001 | 0.0008 | 275.1 |
| | SD | 10.83 | 10.33 | | | | | | |
| | | Placebo | | | | | | | |
| | | D 0 | D 28 | ΔD 28-D 0 | % D 28-D 0 | % respondents | p value D 0 vs D 28 | | |
| | Mean | 43.13 | 44.31 | 1.17 | 2.72 | 50% | 0.1029 | | |
| | SD | 10.75 | 10.00 | | | | | | |

### Biochemical Evaluations

The following biochemical assessments were performed:

From the swab sample:
Natural moisturizing factors (NMFs) by liquid chromatography coupled with UV detection (LC/UV). The amount of NMFs includes urocanic acid (UCA), pyrrolidone carboxylic acid (PCA) and serine. The NMF content provides information on the skin's hydration status.
Ceramides by liquid chromatography coupled with mass spectroscopy detection (LC/MS). The amount of ceramides includes ceramides with [S], [DS] and [P] bases. The ceramide content provides information on the state of the skin barrier.
Inflammatory state via the quantification of cytokines (by ELISA):
   IL1RA
   IL1α
   IL8

From the D-Squames sample:
Nile red/involucrin staining

### NMFs

The results obtained for NMFs at D0 and D28 are given in Table 16. The illustration of the changes is given in FIG. 5.

The results obtained show that the amount of NMFs increases significantly between D0 and D28 with the active agent, whereas the placebo significantly decreases this amount. The comparison of the change between D0 and D28 observed with the active agent versus the change observed with the placebo is statistically significant in favor of the active agent.

TABLE 16
Values at D 0 and D 28 of the amount of NMFs. Percentage changes and statistics.

| | | | | | | | | p value | % Active |
|---|---|---|---|---|---|---|---|---|---|
| | | Active agent | | | | | | Active agent | agent vs |
| NMFs | | D 0 | D 28 | ΔD 28-D 0 | % D 28-D 0 | % respondents | p value D 0 vs D 28 | vs Placebo | Placebo |
| | Mean | 249.51 | 277.72 | 28.21 | 11.31 | 90% | 0.0016 | 0.0005 | 234.7 |
| | SD | 82.30 | 87.86 | | | | | | |
| | | Placebo | | | | | | | |
| | | D 0 | D 28 | ΔD 28-D 0 | % D 28-D 0 | % respondents | p value D 0 vs D 28 | | |
| | Mean | 256.16 | 235.22 | −20.94 | −8.17 | 30% | 0.0049 | | |
| | SD | 92.23 | 83.73 | | | | | | |

### Ceramides

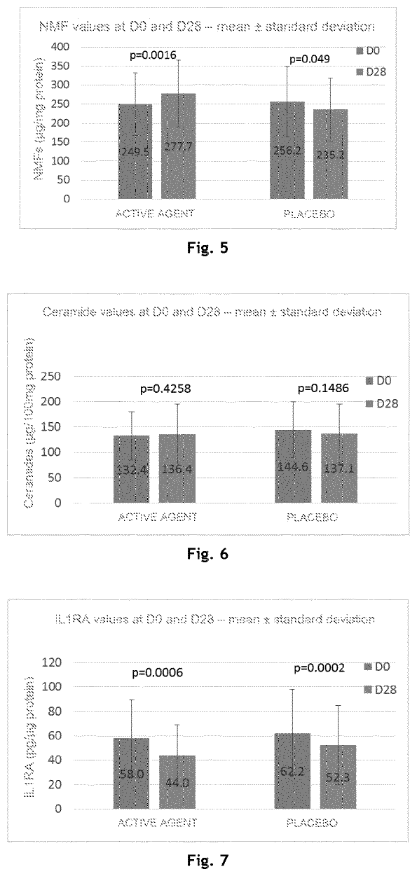

The results obtained for ceramides at D0 and D28 are given in Table 17. The illustration of the changes is given in FIG. 6.

The results obtained show that the amount of ceramides decreases with the placebo, and that the active agent compensates for this decrease (the changes between D0 and D28 for the placebo and the active agent are not significant). The comparison of the change between D0 and D28 observed with the active agent compared with the change observed with the placebo is statistically significant in favor of the active agent.

TABLE 17
Values at D 0 and D 28 of the amount of ceramides. Percentage changes and statistics.

| | | | | | | | | p value | % Active |
|---|---|---|---|---|---|---|---|---|---|
| | | Active agent | | | | | | Active agent | agent vs |
| Ceramides | | D 0 | D 28 | ΔD 28-D 0 | % D 28-D 0 | % respondents | p value D 0 vs D 28 | vs Placebo | Placebo |
| | Mean | 132.44 | 136.42 | 3.98 | 3.01 | 40% | 0.4258 | 0.0188 | 152.6 |
| | SD | 47.28 | 59.03 | | | | | | |
| | | Placebo | | | | | | | |
| | | D 0 | D 28 | ΔD 28-D 0 | % D 28-D 0 | % respondents | p value D 0 vs D 28 | | |
| | Mean | 144.64 | 137.07 | −7.57 | −5.23 | 40% | 0.1486 | | |
| | SD | 55.83 | 57.75 | | | | | | |

### IL1RA

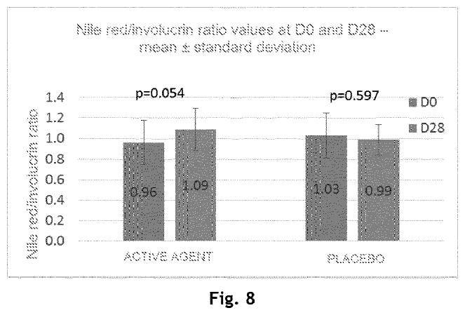

The results obtained for IL1RA at D0 and D28 are given in Table 18. The illustration of the changes is given in FIG. 7.

The results obtained show that the amount of IL1RA decreases significantly between D0 and D28 with the active agent and the placebo. The comparison of the change between D0 and D28 observed with the active agent versus the change observed with the placebo is statistically significant in favor of the active agent.

TABLE 18
Values at D 0 and D 28 of the amount of IL1RA. Percentage changes and statistics.

| | | | | | | | | p value | % Active |
|---|---|---|---|---|---|---|---|---|---|
| | | Active agent | | | | | | Active agent | agent vs |
| IL1RA | | D 0 | D 28 | ΔD 28-D 0 | % D 28-D 0 | % respondents | p value D 0 vs D 28 | vs Placebo | Placebo |
| | Mean | 58.05 | 44.00 | −14.04 | −24.19 | 100% | 0.0006 | 0.0452 | −41.8 |
| | SD | 31.38 | 24.76 | | | | | | |
| | | Placebo | | | | | | | |
| | | D 0 | D 28 | ΔD 28-D 0 | % D 28-D 0 | % respondents | p value D 0 vs D 28 | | |
| | Mean | 62.20 | 52.30 | −9.90 | −15.92 | 100% | 0.0002 | | |
| | SD | 35.54 | 32.20 | | | | | | |

### Nile Red/Involucrin Ratio

The results obtained for the Nile red/involucrin ratio at D0 and D28 are given in Table 19. The illustration of the changes is given in FIG. 8.

The results obtained show that the Nile red/involucrin ratio does not vary significantly between D0 and D28 with the active agent and for the placebo. The comparison of the change between D0 and D28 observed with the active agent versus the change observed with the placebo is statistically significant in favor of the active agent.

TABLE 19
Values at D 0 and D 28 of the Nile red/involucrin ratio. Percentage changes and statistics.

| | | | | | | | | p value | % Active |
|---|---|---|---|---|---|---|---|---|---|
| Nile red/ | | Active agent | | | | | | Active agent | agent vs |
| involucrin ratio | | D 0 | D 28 | ΔD 28-D 0 | % D 28-D 0 | % respondents | p value D 0 vs D 28 | vs Placebo | Placebo |
| | Mean | 0.962 | 1.090 | 0.13 | 13.37 | 80% | 0.0540 | 0.016 | 414.7 |
| | SD | 0.219 | 0.203 | | | | | | |
| | | Placebo | | | | | | | |
| | | D 0 | D 28 | ΔD 28-D 0 | % D 28-D 0 | % respondents | p value D 0 vs D 28 | | |
| | Mean | 1.029 | 0.988 | −0.04 | −3.97 | 70% | 0.5970 | | |
| | SD | 0.219 | 0.152 | | | | | | |

1.-16. (canceled)

17. A peptide extract of microalga Chlamydomonas acidophila obtainable by a process comprising at least one enzymatic hydrolysis step, said peptide extract comprising at least 20% by weight of peptides, the percentages being expressed with respect to the total weight of said extract.

18. The extract as claimed in claim 17, comprising from 20% to 90%, by weight of peptides.

19. The extract as claimed in claim 18, wherein it comprises from 20% to 75%, by weight of peptides.

20. The extract as claimed in claim 19, wherein it comprises from 30% to 70%, by weight of peptides.

21. The extract as claimed in claim 17, wherein the peptides have a molecular weight of less than 3500 Daltons (Da).

22. The extract as claimed in claim 17, wherein at least 80% by weight of the peptides have a molecular weight of less than 1000 Da.

23. The extract as claimed in claim 17, wherein at least 30% by weight of the peptides have a molecular weight of less than 500 Da.

24. A process for preparing a peptide extract of Chlamydomonas acidophila, comprising at least one enzymatic hydrolysis step.

25. The process as claimed in claim 24, wherein the enzymatic hydrolysis step is performed in the presence of at least one protease.

26. The process as claimed in claim 25, wherein the protease is an alkaline protease.

27. The process as claimed in claim 25, comprising:
a) aqueous phase dispersion of the microalga Chlamydomonas acidophila;
b) enzymatic hydrolysis of the aqueous dispersion obtained in step a),
c) heat treatment of the mixture obtained in step b); and
d) recovery of the peptide extract obtained in step c).

28. The process as claimed in claim 27, further comprising an additional filtration or centrifugation step between steps c) and d), optionally followed by ultrafiltration, diafiltration, or nanofiltration.

29. The process as claimed in claim 28, further comprising a step of 15 kDa ultrafiltration between steps c) and d), or after the additional filtration or centrifugation step.

30. The process as claimed in claim 29, further comprising a nanofiltration step with a cutoff between 100 Daltons and 300 Daltons, performed after the 15 kDa ultrafiltration step.

31. A composition, comprising:
a peptide extract of microalga Chlamydomonas acidophila obtainable by a process according to claim 24, as an active principle; and
a suitable excipient.

32. The composition as claimed in claim 31, wherein:
said peptide extract is a peptide extract of microalga Chlamydomonas acidophila obtainable by a preparation process comprising at least one enzymatic hydrolysis step, said peptide extract comprising at least 20% by weight of peptides, the percentages being expressed with respect to the total weight of said extract; or
said peptide extract is obtainable by a process for preparing a peptide extract of Chlamydomonas acidophila, comprising at least one enzymatic hydrolysis step.

33. The composition as claimed in 32, comprising 0.001% to 10% of said peptide extract of Chlamydomonas acidophila, by weight expressed as dry extract, based on the total weight of the composition.

34. The composition as claimed in 33, comprising 0.01% to 5% of said peptide extract of Chlamydomonas acidophila.

35. A method for improving condition or appearance of skin, skin appendages, or mucous membranes, or for treating:
disorders or pathologies of the skin and/or mucous membranes and/or skin appendages, advantageously allergic, inflammatory, irritative reactions or pathologies or disorders of the barrier or homeostasis of the skin, immature, normal, or mature/aged skin appendages and/or mucous membranes, and/or
vascular disorders, in particular redness or couperosis, and/or
alterations of the adipose tissue,
comprising administration of an effective amount of a peptide extract of microalga Chlamydomonas acidophila and a suitable excipient, wherein said peptide extract:
is obtainable by a preparation process comprising at least one enzymatic hydrolysis step, comprising at least 20% by weight of peptides, the percentages being expressed with respect to the total weight of said extract, or
is obtainable by a process comprising at least one enzymatic hydrolysis step.
